(19) United States
(12) Patent Application Publication
(10) Pub. No.: US 2024/0165094 A1
(43) Pub. Date: May 23, 2024

(54) METHODS AND COMPOSITIONS FOR TREATING MELANOMA

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE (CNRS), Paris (FR); UNIVERSITE COTE D'AZUR, Nice (FR)

(72) Inventors: Marcel DECKERT, Nice Cedex 3 (FR); Sophie TARTARE-DECKERT, Nice Cedex3 (FR); Bernard MARI, Sophia-Antipolis (FR); Serena DIAZZI, Nice Cedex 03 (FR)

(21) Appl. No.: 18/550,352

(22) PCT Filed: Mar. 16, 2022
(86) PCT No.: PCT/EP2022/056776

§ 371 Date: Sep. 13, 2023

(30) Foreign Application Priority Data
Mar. 17, 2021 (EP) 21305325.9

(51) Int. Cl. A61K 31/4523; A61K 31/437; A61K 31/496; A61K 31/506; A61K 31/519; A61K 45/06; A61P 35/00

(52) U.S. Cl. CPC A61K 31/4523; A61K 31/437; A61K 31/496; A61K 31/506; A61K 31/519; A61K 45/06; A61P 35/00

(57) ABSTRACT

The inventors' working hypothesis relies on the original observations that mesenchymal MAPKi resistant cells display markers of fibrosis, acquire a myofibroblast-like phenotype and extracellular matrix (ECM) remodelling activities. In addition to increased remodelling of the ECM, pro-fibrotic responses induced by MAPK-targeted therapy include enhanced actin cytoskeleton plasticity, high sensitivity to mechanical cues and the establishment of an inflammatory microenvironment that contribute to therapy escape. The inventor's reason that approaches aimed at manipulating this abnormal fibrotic-like response induced by targeted therapy may represent rationale combination strategies to normalize the fibrous stroma, enhance drug efficacy and overcome resistance in BRAE mutant melanoma. Here the inventors investigated the impact of Nintedanib, an EMA/FDA-approved anti-fibrotic drug, as a repurposed drus in combination with targeted therapy on melanoma cell viability and tumor growth. Their findings reveal that the triplet combination BRAFi/MEKi/Nintedanib is active in pre-clinical models of melanoma to normalize the fibrous ECM network, enhance the efficacy of MAPK-targeted therapy and delay tumor relapse. Accordingly, the invention relates to a method for treating melanoma in a subject in need thereof comprising a step of administering said subject with a therapeutically effective amount of: i) an inhibitor of BRAE, ii) an inhibitor of MEK and iii) an anti-fibrotic agent.

### FIELD OF THE INVENTION

The invention is in the field of oncology. More particularly, the invention relates to methods and compositions to treat melanoma.

### BACKGROUND OF THE INVENTION

Melanoma is the leading cause of skin cancer deaths worldwide. The majority of malignant melanomas harbor activating mutations in the BRAF oncogene, leading to hyper activation of the mitogen-activated protein kinase (MAPK) signaling pathway. Targeting this driver mutation using BRAF inhibitors (BRAFi) such as Vemurafenib or Dabrafenib combined to MEK inhibitors (MEKi) such as Cobimetinib or Trametinib has markedly improved the clinical outcome of patients with BRAFV600E/K mutant melanoma (Robert et al., 2019). Despite this, therapy resistance is nearly universal and is regarded as a major obstacle for achieving complete cure.

Thus, there is an unmet need for new strategies that would prevent and/or delay resistance of melanoma cells to MAPK-targeted therapy. Such therapeutic approaches will benefit to patients and will lower the risk of relapse and metastatic recurrence.

Recent preclinical studies and translational efforts have provided insights into mechanisms of upfront and acquired resistance to BRAF and MEK inhibitors. Acquired resistance to targeted drugs involves genetic alterations and non-genetic alterations that are largely driven by tumor cell plasticity and phenotype switching processes (Marine et al., 2020). Upon drug pressure, melanoma cells have the ability to re-activate developmental programs and switch to a dedifferentiated mesenchymal-like state characterized by upregulation of receptor tyrosine kinases (RTK) like PDGFR, NGFR or AXL and downregulation of melanocytic differentiation proteins such as MITF (Rambow et al., 2019; Arozarena and Wellbrock, 2019). This cell state is associated with a greater metastatic potential. Importantly, such adaptive responses to BRAF oncogenic pathway inhibition are thought to precede the emergence of mutation-driven acquired resistance.

The inventors' working hypothesis relies on the original observations that mesenchymal MAPKi resistant cells display markers of fibrosis, acquire a myofibroblast-like phenotype and extracellular matrix (ECM) remodeling activities (Diazzi et al., 2020; Girard et al., 2020). In addition to increased remodeling of the ECM, pro-fibrotic responses induced by MAPK-targeted therapy include enhanced actin cytoskeleton plasticity, high sensitivity to mechanical cues and the establishment of an inflammatory microenvironment that contribute to therapy escape. The inventor's reason that approaches aimed at manipulating this abnormal fibrotic-like response induced by targeted therapy may represent rationale combination strategies to normalize the fibrous stroma, enhance drug efficacy and overcome resistance in BRAF mutant melanoma.

Here the inventors investigated the impact of Nintedanib (BIBF1120, Intedanib, Ofev, Vargatef), an EMA/FDA-approved anti-fibrotic drug, as a repurposed drug in combination with targeted therapy on melanoma cell viability and tumor growth. Their findings reveal that the triplet combination BRAFi/MEKi/Nintedanib is active in pre-clinical models of melanoma to normalize the fibrous ECM network, enhance the efficacy of MAPK-targeted therapy and delay tumor relapse.

### SUMMARY OF THE INVENTION

The invention relates to a method for treating melanoma in a subject in need thereof comprising a step of administering said subject with a therapeutically effective amount of: i) an inhibitor of BRAF, ii) an inhibitor of MEK, and iii) an anti-fibrotic agent. In particular, the invention is defined by claims.

### DETAILED DESCRIPTION OF THE INVENTION

The majority of melanomas harbor BRAF activating mutations, leading to hyper activation of the MAPK signaling pathway. Targeting this driver mutation using BRAF inhibitors combined to MEK inhibitors has markedly improved the clinical outcome of patients. However, therapy resistance is nearly universal and is regarded as a major obstacle for achieving complete cure. Acquired resistance involves genetic and non-genetic alterations that are largely driven by tumor cell plasticity and phenotype switching. Upon drug pressure, melanoma cells have the ability to re-activate developmental programs and switch to a dedifferentiated mesenchymal-like state associated with a greater metastatic potential. The inventor's working hypothesis relies on the previous observations that mesenchymal resistant cells display markers of fibrosis, acquire a myofibroblast-like phenotype and extracellular matrix (ECM) remodeling activities. In addition to increased ECM remodeling, pro-fibrotic responses induced by MAPK-targeted therapy include the establishment of an inflammatory stroma that contribute to therapy escape. The inventor's reason that approaches aimed at manipulating this abnormal fibrotic-like response induced by targeted therapy may represent rationale combination strategies to normalize the fibrous stroma, enhance drug efficacy and overcome resistance in BRAF-mutant melanoma. They set out to analyze the effects of the anti-fibrotic drug Nintedanib in the context of early adaptation and resistance to MAPK-targeted therapy in melanoma cells.

Accordingly, the invention relates to a method for treating melanoma in a subject in need thereof comprising a step of administering said subject with a therapeutically effective amount of: i) an inhibitor of BRAF, ii) an inhibitor of MEK and iii) an anti-fibrotic agent.

In another embodiment, the invention relates to a method for treating melanoma in a subject in need thereof comprising a step of administering said subject with a therapeutically effective amount of: i) an inhibitor of MEK and ii) an anti-fibrotic agent.

In another embodiment, the invention relates to a method for treating melanoma in a subject in need thereof comprising a step of administering said subject with a therapeutically effective amount of : i) an inhibitor of BRAF and ii) an anti-fibrotic agent.

Accordingly, the invention relates to a method for treating resistant melanoma in a subject in need thereof comprising a step of administering said subject with a therapeutically effective amount of: i) an inhibitor of BRAF, ii) an inhibitor of MEK and iii) an anti-fibrotic agent.

In another embodiment, the invention relates to a method for treating resistant melanoma in a subject in need thereof comprising a step of administering said subject with a therapeutically effective amount of: i) an inhibitor of MEK and ii) an anti-fibrotic agent.

In another embodiment, the invention relates to a method for treating resistant melanoma in a subject in need thereof comprising a step of administering said subject with a therapeutically effective amount of : i) an inhibitor of BRAF and ii) an anti-fibrotic agent.

As used herein, the term “subject” denotes a mammal, such as a rodent, a feline, a canine, and a primate. Particularly, the subject according to the invention is a human. More particularly, the subject according to the invention has or is susceptible to have melanoma. In particular embodiment, the subject has or is susceptible to have cutaneous melanoma. In a particular embodiment, the subject has or is susceptible to have melanoma resistant. In a particular embodiment, the subject has or is susceptible to have BRAFV600E/K mutation.

As used herein, the term “melanoma” also known as malignant melanoma, refers to a type of cancer that develops from the pigment-containing cells, called melanocytes. There are three general categories of melanoma: 1) cutaneous melanoma which corresponds to melanoma of the skin; it is the most common type of melanoma; 2) mucosal melanoma which can occur in any mucous membrane of the body, including the nasal passages, the throat, the vagina, the anus, or in the mouth; and 3) ocular melanoma also known as uveal melanoma or choroidal melanoma, is a rare form of melanoma that occurs in the eye.

In a particular embodiment, the melanoma is cutaneous melanoma.

In another embodiment, the melanoma is resistant melanoma.

As used herein, the term “resistant melanoma” refers to melanoma, which does not respond to a classical treatment. The cancer may be resistant at the beginning of treatment or it may become resistant during treatment. The resistance to drug leads to rapid progression of metastatic of melanoma. The resistance of cancer for the medication is caused by mutations in the gene, which are involved in the proliferation, divisions or differentiation of cells. In the context of the invention, the resistance of melanoma is caused by the mutations (single or double) in the following genes: BRAF, MEK, NRAS or PTEN. The resistance can be also caused by a double-negative BRAF and NRAS mutation. The NRAS gene is in the Ras family of oncogene and involved in regulating cell division. NRAS mutations in codons 12, 13, and 61 arise in 15-20% of all melanomas. The inhibitors of BRAF mutation or MEK are used to treat the melanoma with NRAS mutations. In a particular embodiment, the melanoma is resistant in which double-negative BRAF and NRAS mutant melanoma. The term “PTEN” refers to Phosphatase and TENsin homolog, it is one of the most frequently inactivated tumor suppressor genes in sporadic cancers. Inactivating mutations and deletions of the PTEN gene are found in many types of cancers, including melanoma. The resistance can be also caused by a triple-negative NRAS, BRAF and MEK mutations as described above. Accordingly, such resistance is against to the treatments as described above.

As used herein, the term “classical treatment” refers to any compound, natural or synthetic, used for the treatment of a melanoma. In a particular embodiment, the classical treatment refers to radiation therapy, antibody therapy or chemotherapy.

As used herein, the term “chemotherapy” has its general meaning in the art and refers to chemical compounds that are effective in inhibiting tumor growth. Examples of chemotherapeutic agents include multikinase inhibitors such as sorafenib and sunitinib, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33: 183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino- doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti- adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®, razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisp latin and carbop latin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

As used herein, the term “targeted therapy” refers to drugs which attack specific genetic mutations within cancer cells, such as melanoma while minimising harm to healthy cells. Typically, the targeted therapy for melanoma refers to use of BRAF, MEK or NRAS inhibitors as described above.

As used herein, the term “radiation therapy” has its general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

In another embodiment, the melanoma is resistant to a combined treatment.

As used herein, the terms “combined treatment”, “combined therapy” or “therapy combination” refer to a treatment that uses more than one medication. The combined therapy may be dual therapy or bi-therapy. In the context of the invention, the melanoma is resistant to a combined treatment characterized by using an inhibitor of BRAF mutation and an inhibitor of MEK as described above. For example, the combined treatment may be a combination of Vemurafenib and Cotellic.

In a further embodiment, the melanoma is resistant to a treatment with an immune checkpoint inhibitor.

As used herein, the term “immune checkpoint inhibitor” refers to molecules that totally or partially reduce, inhibit, interfere with or modulate one or more immune checkpoint proteins. As used herein, the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480- 489). Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and plays a role in tumour escape. B and T Lymphocyte Attenuator (BTLA) and also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. A related immune-inhibitory enzymes. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA, Short for V-domain Ig suppressor of T cell activation, VISTA is primarily expressed on hematopoietic cells so that consistent expression of VISTA on leukocytes within tumors may allow VISTA blockade to be effective across a broad range of solid tumors. Tumor cells often take advantage of these checkpoints to escape detection by the immune system. Thus, inhibiting a checkpoint protein on the immune system may enhance the anti-tumor T-cell response.

In some embodiments, an immune checkpoint inhibitor refers to any compound inhibiting the function of an immune checkpoint protein. Inhibition includes reduction of function and full blockade. In some embodiments, the immune checkpoint inhibitor could be an antibody, synthetic or native sequence peptides, small molecules or aptamers which bind to the immune checkpoint proteins and their ligands.

In a particular embodiment, the immune checkpoint inhibitor is an antibody.

Typically, antibodies are directed against A2AR, B7-H3, B7-H4, BTLA, CTLA-4, CD277, IDO, KIR, PD-1, LAG-3, TIM-3 or VISTA.

In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (Opdivo®, BMS), Pembrolizumab (also called Lambrolizumab, KEYTRUDA® or MK-3475, MERCK).

In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat. No. 8,617,546 and WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS).

In some embodiments, the immune checkpoint inhibitor is an anti-PD-L2 antibody such as described in U.S. Pat. Nos. 7,709,214, 7,432,059 and 8,552,154.

In the context of the invention, the immune checkpoint inhibitor inhibits Tim-3 or its ligand.

In a particular embodiment, the immune checkpoint inhibitor is an anti-Tim-3 antibody such as described in WO03063792, WO2011155607, WO2015117002, WO2010117057 and WO2013006490.

In some embodiments, the immune checkpoint inhibitor is a small organic molecule.

The term “small organic molecule” as used herein, refers to a molecule of a size comparable to those organic molecules generally used in pharmaceuticals. The term excludes biological macro molecules (e. g. proteins, nucleic acids, etc.). Typically, small organic molecules range in size up to about 5000 Da, more preferably up to 2000 Da, and most preferably up to about 1000 Da.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with Indoleamine-pyrrole 2,3-dioxygenase (IDO) inhibitor. IDO is involved in the tryptophan catabolism (Liu et al 2010, Vacchelli et al 2014, Zhai et al 2015). Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. In a particular embodiment, the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In a particular embodiment, the inhibitor of IDO is Epacadostat, (INCB24360, INCB024360) has the following chemical formula in the art and refers to —N-(3-bromo-4-fluorophényl)-N′-hydroxy-4-{[2-(sulfamoylamino)-éthyl]amino}-1,2,5-oxadiazole-3 carboximidamide:

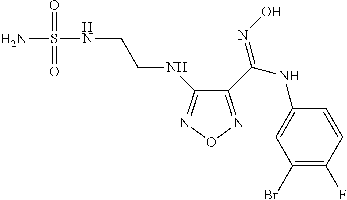 [SMILES: NS(=O)(=O)NCCNc1nonc1/C(=N/O)Nc1ccc(F)c(Br)c1]

In a particular embodiment, the inhibitor is BGB324, also called R428, such as described in WO2009054864, refers to 1H-1,2,4-Triazole-3,5-diamine, 1-(6,7-dihydro-5H-benzo[6,7]cyclohepta[1,2-c]pyridazin-3-yl)-N3-[(7S)-6,7,8,9-tetrahydro-7-(1-pyrrolidinyl)-5H-benzocyclohepten-2-yl]- and has the following formula in the art:

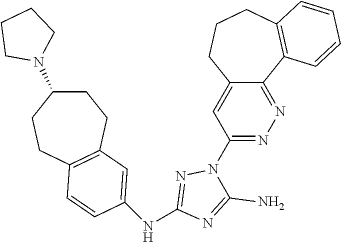 [SMILES: Nc1nc(Nc2ccc3c(c2)CC[C@@H](N2CCCC2)CC3)nn1-c1cc2c(nn1)-c1ccccc1CCC2]

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

In a further embodiment, the melanoma is resistant to a treatment with the inhibitors of BRAF.

As used herein, the term “BRAF” is a member of the Raf kinase family of serine/threonine-specific protein kinases. This protein plays a role in regulating the MAP kinase/ERKs signaling pathway, which affects cell division, differentiation, and survival. A number of mutations in BRAF are known. In particular, the V600E mutation is prominent. Other mutations which have been found are R461I, I462S, G463E, G463V, G465A, G465E, G465V, G468A, G468E, N580S, E585K, D593V, F594L, G595R, L596V, T598I, V599D, V599E, V599K, V599R, K600E, A727V, and most of these mutations are clustered to two regions: the glycine-rich P loop of the N lobe and the activation segment and flanking regions. In a particular embodiment, the BRAF mutation is V600E/K in the context of the invention. As used herein, the term “inhibitor of BRAF” refers to a natural or synthetic compound that has a biological effect to inhibit the activity or the expression of BRAF. More particularly, such compound by inhibiting BRAF activity reduces cell division, differentiation, and secretion. In a particular embodiment, the inhibitor of BRAF is a peptide, peptidomimetic, small organic molecule, antibody, aptamers, siRNA or antisense oligonucleotide. The term “peptidomimetic” refers to a small protein-like chain designed to mimic a peptide. In a particular embodiment, the inhibitor of BRAF is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity.

In a particular embodiment, the inhibitor of BRAF is a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da. The inhibitors of BRAF mutations are well known in the art. In a particular embodiment, the inhibitor of BRAF is Vemurafenib. Vemurafenib also known as PLX4032, RG7204 or RO5185426 and commercialized by Roche as zelboraf. In a particular embodiment, the inhibitor of BRAF is Dabrafenib also known as tafinlar, which is commercialized by Novartis. In a particular embodiment, the melanoma is resistant to a treatment with dacarbazine. Dacarbazine also known as imidazole carboxamide is commercialized as DTIC-Dome by Bayer.

In a further embodiment, the melanoma is resistant to a treatment with the inhibitors of MEK.

As used herein, the term “MEK” refers to Mitogen-activated protein kinase, also known as MAP2K, MEK, MAPKK. It is a kinase enzyme, which phosphorylates mitogen-activated protein kinase (MAPK). As used herein, the term “inhibitor of MEK” refers to a natural or synthetic compound that has a biological effect to inhibit the activity or the expression of MEK. More particularly, such compound by inhibiting BRAF activity reduces phosphorylation of MAPK. In a particular embodiment, the inhibitor of MEK is a peptide, peptidomimetic, small organic molecule, antibody, aptamers, siRNA or antisense oligonucleotide.

In a particular embodiment, the inhibitor of MEK is a small organic molecule.

The inhibitors of MEK are well known in the art. In a particular embodiment, the inhibitor of MEK is Trametinib also known as mekinist, which is commercialized by GSK. In a particular embodiment, the inhibitor of MEK is Cobimetinib also known as cotellic commercialized by Genentech. In a particular embodiment, the inhibitor of MEK is Binimetinib also known as MEK162, ARRY-162 is developed by Array Biopharma.

In a particular embodiment, the melanoma is resistant to a treatment with the inhibitors of NRAS. The NRAS gene is in the Ras family of oncogene and involved in regulating cell division. NRAS mutations in codons 12, 13, and 61 arise in 15-20% of all melanomas. The inhibitors of BRAF mutation or MEK are used to treat the melanoma with NRAS mutations. In a particular embodiment, the melanoma is resistant in which double-negative BRAF and NRAS mutant melanoma.

As used herein, the term “fibrosis”, also known as fibrotic scarring, refers to a pathological wound healing in which connective tissue replaces normal parenchymal tissue to the extent that it goes unchecked, leading to considerable tissue remodelling and the formation of permanent scar tissue. Repeated injuries, chronic inflammation and repair are susceptible to fibrosis where an accidental excessive accumulation of extracellular matrix components, such as the collagen is produced by fibroblasts, leading to the formation of a permanent fibrotic scar. In response to injury, this is called scarring, and if fibrosis arises from a single cell line, this is called a fibroma. Physiologically, fibrosis acts to deposit connective tissue, which can interfere with or totally inhibit the normal architecture and function of the underlying organ or tissue. Fibrosis can be used to describe the pathological state of excess deposition of fibrous tissue, as well as the process of connective tissue deposition in healing. Defined by the pathological accumulation of extracellular matrix (ECM) proteins, fibrosis results in scarring and thickening of the affected tissue, it is in essence an exaggerated wound healing response which interferes with normal organ function.

As used herein, the term “anti-fibrotic agent” refers to an agent that inhibits or reduces fibrosis.

Examples of anti-fibrotic agents include, without limitation, TGF-beta neutralizing antibodies, angiotension II inhibitors, inhibitors of fibrogenic cytokine signaling (e.g., TNF-alpha neutralizing antibodies, and inhibitors or neutralizing antibodies against IL6 signaling), integrin inhibitors, and other small molecules or inhibitors or neutralizing antibodies that may directly target fibrogenic signaling, indirectly target fibrogenic signaling (e.g., upstream or downstream modulators), or suppress fibrogenic signaling. Specific examples of anti-fibrotic agents include, without limitation, SHP-627, hydronidone PXS-25, disitertide, fresolimumab, LY2382770, STX-100, CWHM-12, SB-431542, THR-184, PF-06473871, RXI-109, FG-3019, imatinib, BOT-191, nilotinib, dasatinib, nintedanib, sorafenib, thalidomide, pomalidomide, etanercept, belimumab, refanalin (BB-3), dectrekumab (Q AX-576), tralokinumab, anakinra, rilonacept, SAR156597, carlumab, bindarit, maraviroc, RS-504393, actimmune, IFN-a, batimastat (BB-49), marimastat, macitentan, bosentan, ambrisentan, sparsentan (RE-021), atrasentan, losartan, BMS-986020, SAR-100842, PART antagonists, curcumin, silymarin, b-caryophyllene, beraprost, iloprost, treprostinil, aviptadil, sivelestat, UK-396082, serelaxin, PRM-151, dioscin, NTU281, rapamycin, palomid-529 (RES-529), ruxolitinib, baricitinib, omipalisib (GSK2126458), PF-562271, tanzisertib (CC-930), MMI-0100, IMD-1041, bardoxolone methyl (CDDO-Me), antisense NF-KB, baicalein, sulfasalazine, Y-27632, bortezomib, emricasan, VX-166, Z-VAD-fmk, CTP-499, VBY-376, CA-074Me, paquinimod, HOE-077, rosiglitazone, elafibranor (GFT-505), saroglitazar, pioglitazone, docosahexaenoic acid, INT-767, PX-102, obeti cholic acid, turofexorate isopropyl (WAY-362450), GW4064, triamcinolone, genistein, pentoxifylline, SIS-3, glycyrrhizin, anti-miR-21, ademetionine (SAM), b-aminopropionitrile (BAPN), simtuzumab (GS-6624), GM-CT-01, GR-MD-02, GCS-100, GKT137831, N-acetylcysteine, mitoquinone, salvianolic acid B, resveratrol, pyridoxamine, a-tocopherol, and IW001.

In a particular embodiment, the anti-fibrotic agent is nintedanib.

In some embodiments, the inhibitor of BRAF or MEK expression is a short hairpin RNA (shRNA), a small interfering RNA (siRNA) or an antisense oligonucleotide, which inhibits the expression of BRAF or MEK. In a particular embodiment, the inhibitor of BRAF or MEK expression is siRNA. A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound. Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway whereby the siRNA interferes with the expression of a specific gene. Anti-sense oligonucleotides include anti-sense RNA molecules and anti-sense DNA molecules, would act to directly block the translation of the targeted mRNA by binding thereto and thus preventing protein translation or increasing mRNA degradation, thus decreasing the level of the targeted protein, and thus activity, in a cell. For example, antisense oligonucleotides of at least about 15 bases and complementary to unique regions of the mRNA transcript sequence can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Antisense oligonucleotides, siRNAs, shRNAs of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically mast cells. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

In some embodiments, the inhibitor of BRAF or MEK expression is an endonuclease. In the last few years, staggering advances in sequencing technologies have provided an unprecedentedly detailed overview of the multiple genetic aberrations in cancer. By considerably expanding the list of new potential oncogenes and tumor suppressor genes, these new data strongly emphasize the need of fast and reliable strategies to characterize the normal and pathological function of these genes and assess their role, in particular as driving factors during oncogenesis. As an alternative to more conventional approaches, such as cDNA overexpression or downregulation by RNA interference, the new technologies provide the means to recreate the actual mutations observed in cancer through direct manipulation of the genome. Indeed, natural and engineered nuclease enzymes have attracted considerable attention in the recent years. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homology-directed repair (HDR).

In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences.

In some embodiment, the endonuclease is CRISPR-cas9 which is from Streptococcus pyogenes. The CRISPR/Cas9 system has been described in U.S. PAt. No. 8,697,359 B1 and US 2014/0068797. Originally an adaptive immune system in prokaryotes (Barrangou and Marraffini, 2014), CRISPR has been recently engineered into a new powerful tool for genome editing. It has already been successfully used to target important genes in many cell lines and organisms, including human (Mali et al., 2013, Science, Vol. 339 : 823-826), bacteria (Fabre et al., 2014, PLoS Negl. Trop. Dis., Vol. 8:e2671.), zebrafish (Hwang et al., 2013, PLoS One, Vol. 8:e68708.), C. elegans (Hai et al., 2014 Cell Res. doi: 10.1038/cr.2014.11.), bacteria (Fabre et al., 2014, PLoS Negl. Trop. Dis., Vol. 8:e2671.), plants (Mali et al., 2013, Science, Vol. 339: 823-826), Xenopus tropicalis (Guo et al., 2014, Development, Vol. 141 : 707-714.), yeast (DiCarlo et al., 2013, Nucleic Acids Res., Vol. 41 : 4336-4343.), Drosophila (Gratz et al., 2014 Genetics, doi:10.1534/genetics.113.160713), monkeys (Niu et al., 2014, Cell, Vol. 156 : 836-843.), rabbits (Yang et al., 2014, J. Mol. Cell Biol., Vol. 6: 97-99.), pigs (Hai et al., 2014, Cell Res. doi: 10.1038/cr.2014.11.), rats (Ma et al., 2014, Cell Res., Vol. 24: 122-125.) and mice (Mashiko et al., 2014, Dev. Growth Differ. Vol. 56: 122-129.). Several groups have now taken advantage of this method to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. A recent exciting development is the use of the dCas9 version of the CRISPR/Cas9 system to target protein domains for transcriptional regulation, epigenetic modification, and microscopic visualization of specific genome loci.

In some embodiment, the endonuclease is CRISPR-Cpf1, which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

In some embodiments, the inhibitor of BRAF or MEK is an antibody. As used herein, the term “antibody” is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. The term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001 ; Reiter et al., 1996; and Young et al., 1995 further describe and enable the production of effective antibody fragments. In some embodiments, the antibody is a “chimeric” antibody as described in U.S. Pat. No. 4,816,567. In some embodiments, the antibody is a humanized antibody, such as described U.S. Pat. Nos. 6,982,321 and 7,087,409. In some embodiments, the antibody is a human antibody. A “human antibody” such as described in U.S. PAt. No. 6,075,181 and 6,150,584. In some embodiments, the antibody is a single domain antibody such as described in EP 0 368 684, WO 06/030220 and WO 06/003388.

In a particular embodiment, the inhibitor of BRAF or MEK is a monoclonal antibody. Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique.

In a particular embodiment, the method according to the invention, wherein, i) the inhibitor of BRAF is Vemurafenib; ii) the inhibitor of MEK is Trametinib and iii) the anti-fibrotic agent is Nintedanib.

In a particular embodiment, the method according to the invention, wherein, i) the inhibitor of BRAF is Vemurafenib; ii) the inhibitor of MEK is Cobimetinib and iii) the anti-fibrotic agent is Nintedanib.

In a particular embodiment, the method according to the invention, wherein, i) the inhibitor of BRAF is Dabrafenib; ii) the inhibitor of MEK is Trametinib and iii) the anti-fibrotic agent is Nintedanib.

In a particular embodiment, the method according to the invention, wherein, i) the inhibitor of BRAF is Dabrafenib; ii) the inhibitor of MEK is Cobimetinib and iii) the anti-fibrotic agent is Nintedanib.

As used herein the terms “administering” or “administration” refer to the act of injecting or otherwise physically delivering a substance as it exists outside the body (e.g., an inhibitor of BRAF, MEK and an anti-fibrotic agent) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

In a further embodiment, the invention relates to i) an inhibitor of BRAF, ii) an inhibitor of MEK and iii) an inhibitor of an anti-fibrotic agent as a combined preparation for simultaneous, separate or sequential use in the treatment of melanoma.

In a further embodiment, the invention relates to i) an inhibitor of BRAF, and ii) an anti-fibrotic agent as a combined preparation for simultaneous, separate or sequential use in the treatment of melanoma.

In a further embodiment, the invention relates to i) an inhibitor of MEK and ii) an anti- fibrotic agent as a combined preparation for simultaneous, separate or sequential use in the treatment of melanoma.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

By a “therapeutically effective amount” is meant a sufficient amount of inhibitor of BRAF, MEK and an anti-fibrotic agent for use in a method for the treatment of melanoma at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The anti-fibrotic agent as described above may be combined with pharmaceutically acceptable excipients, and optionally sustained-release matrices, such as biodegradable polymers, to form pharmaceutical compositions.

A further objective the invention relates to a pharmaceutical composition comprising i) an inhibitor of BRAF, ii) an inhibitor of MEK and iii) an anti-fibrotic agent.

In a particular embodiment, the invention relates to a pharmaceutical composition comprising i) an inhibitor of BRAF, and ii) an anti-fibrotic agent.

In another embodiment, the invention relates to a pharmaceutical composition comprising i) an inhibitor of MEK and iii) an anti-fibrotic agent 2.

In a particular embodiment, the invention refers to a pharmaceutical composition according to the invention, for use in the therapy.

In a particular embodiment, the invention refers to a pharmaceutical composition according to the invention, for use in the treatment of melanoma.

In a particular embodiment, the invention refers to a pharmaceutical composition according to the invention, wherein i) the inhibitor of BRAF is Vemurafenib; ii) the inhibitor of MEK is Trametinib and iii) the anti-fibrotic agent is Nintedanib.

In a particular embodiment, the invention refers to a pharmaceutical composition according to the invention, wherein i) the inhibitor of BRAF is Vemurafenib; ii) the inhibitor of MEK is Cobimetinib and iii) the anti-fibrotic agent is Nintedanib.

In a particular embodiment, the invention refers to a pharmaceutical composition according to the invention, wherein i) the inhibitor of BRAF is Dabrafenib; ii) the inhibitor of MEK is Trametinib and iii) the anti-fibrotic agent is Nintedanib.

In a particular embodiment, the invention refers to a pharmaceutical composition according to the invention, wherein i) the inhibitor of BRAF is Dabrafenib; ii) the inhibitor of MEK is Cobimetinib and iii) the anti-fibrotic agent is Nintedanib.

In one embodiment, the pharmaceutical composition of the invention is administered in combination with a classical treatment of melanoma.

In one embodiment, the inhibitor of BRAF, the inhibitor of MEK and the anti-fibrotic agent of the invention are administered in combination with a classical treatment of melanoma.

In one embodiment, the inhibitor of MEK and the anti-fibrotic agent of the invention are administered in combination with a classical treatment of melanoma.

In one embodiment, the inhibitor of BRAF and the anti-fibrotic agentof the invention are administered in combination with a classical treatment of melanoma.

In particular embodiment, the pharmaceutical composition of the invention is administered in combination with an anti-PD-1 antibody.

In particular embodiment, the inhibitor of BRAF, the inhibitor of MEK and the anti- fibrotic agent of the invention are administered in combination with an anti-PD-1 antibody.

In particular embodiment, the inhibitor of MEK and the anti-fibrotic agent of the invention are administered in combination with an anti-PD-1 antibody.

In particular embodiment, the inhibitor of BRAF and the anti-fibrotic agent of the invention are administered in combination with an anti-PD-1 antibody

The inhibitors of BRAF, MEK and/or the anti-fibrotic agent as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

A further object of the present invention relates to a method of screening a drug suitable for the treatment of melanoma comprising i) providing a test compound and ii) determining the ability of said test compound to inhibit the activity of BRAF or MEK.

Any biological assay well known in the art could be suitable for determining the ability of the test compound to inhibit the activity of BRAF or MEK. In some embodiments, the assay first comprises determining the ability of the test compound to bind to BRAF or MEK. In some embodiments, a population of cells is then contacted and activated so as to determine the ability of the test compound to inhibit the activity of BRAF or MEK. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of inhibiting the activity of BRAF or MEK, as determined using in vitro methods described herein, are likely to exhibit similar modulatory capacity in applications in vivo. Typically, the test compound is selected from the group consisting of peptides, peptidomimetics, small organic molecules, aptamers or nucleic acids. For example the test compound according to the invention may be selected from a library of compounds previously synthesised, or a library of compounds for which the structure is determined in a database, or from a library of compounds that have been synthesised de novo. In some embodiments, the test compound may be selected form small organic molecules.

The invention will be further illustrated by the following figures and examples. However, these examples and figures should not be interpreted in any way as limiting the scope of the present invention.

### FIGURES

FIG. 1. Nintedanib sensitizes melanoma cells to BRAFi/MEKi therapy and prevents BRAFi/MEKi-driven ECM remodeling. (A) BRAFi-resistant M238R cells were treated with vehicle (Ctrl), Nintedanib (2 μM), Vemurafenib and Trametinib (BRAFi/MEKi, 5 μM) or Vemurafenib/Trametinib (5 μM) plus Nintedanib (2 μM) for 3 days. Cell viability was assessed by crystal violet staining of attached cells. (B) BRAFi-sensitive parental M238P cells were treated with vehicle (Ctrl), Nintedanib (2 μM) Vemurafenib and Trametinib (BRAFi/MEKi, 1 μM) or Vemurafenib/Trametinib (1 μM) plus Nintedanib (2 μM) for 3 days. Cell viability was assessed by crystal violet staining of attached cells. Paired Student t-test has been used for statistical analysis. ****P≤0.0001. Data is represented as mean ±SE from a triplicate representative of at least 3 independent experiments.

FIG. 2. Nintedanib enhances MAPK-targeted therapy efficacy, delays tumor relapse, improves mice survival and normalizes the pro-fibrotic stromal reaction. Kaplan-Meier survival curves of mice treated with vehicle, Nintedanib, MAPK-targeted therapy (BRAFi+MEKi) or MAPK-targeted therapy plus Nintedanib. Log rank (Mantel-Cox) statistical test was used for MAPK-targeted therapy vs MAPK targeted therapy/Nintedanib. ****p<0.0001.

FIG. 3. Effect of different BRAFi/MEKi therapeutic combinations, in the presence or not of Nintedanib, on melanoma cell viability. BRAFi-resistant M238R cells were treated with vehicle (Ctrl), Nintedanib (2 μM), Vemurafenib/Cobimetinib (1 μM), Vemurafenib/Cobimetinib (1 μM) plus Nintedanib (2 μM), Dabrafenib/Trametinib (1 μM) or Dabrafenib/Trametinib (1 μM) plus Nintedanib (2 μM) for 3 days. Cell viability was assessed by crystal violet staining of attached cells.

FIG. 4. Nintedanib, but not Pirfenidone, enhances MAPK-targeted therapy efficacy and delays tumor relapse. Mouse YUMM1.7 cells were injected s.c. into syngeneic C57BL/6 mice. When tumor volume reached 100 mm3, mice were administered vehicle, BRAFi+MEKi combination, Nintedanib, Pirfenidone or the triple combinations of BRAFi+MEKi and Nintedanib or BRAFi+MEKi and Pirfenidone. Representative median graphics show tumor growth following treatment (n=3 mice per group). Treatment: Oral gavage 3 times per week. Vemurafenib (BRAFi): 30 mg/kg; Trametinib (MEKi): 0.3 mg/kg; Nintedanib: 50 mg/kg; Pirfenidone: 100 mg/kg. Oral gavage 3 times per week.

### EXAMPLE

### Material & Methods

### Cell Lines and Reagents

The isogenic pair of Vemurafenib-sensitive and -resistant human BRAF-mutated melanoma cells M238 was provided by R. Lo (Nazarian et al., 2010). YUMM1.7 mouse melanoma cells were a kind gift from M. Bosenberg (Meeth et al., 2016). Melanoma cells were cultured in Dulbecco's modified Eagle medium (DMEM) supplemented with 7% FBS (Hyclone) and 1% penicillin/streptomycin. Resistant cells were continuously exposed to 1 μM of Vemurafenib. Cell lines were routinely tested for the absence of Mycoplasma by PCR. Culture reagents were purchased from Thermo Fisher Scientific. BRAFi (PLX4032, Vemurafenib), MEKi (GSK1120212, Trametinib) and Nintedanib (BIBF1120) were from Selleckem.

### Viability Assay

After the indicated treatments, cells were stained with 0.04% crystal violet, 20% ethanol in PBS for 30 min. Following accurate washing of the plate, representative photographs were taken. The crystal violet dye is solubilized by 10% acetic acid in PBS and measured by absorbance at 595 nm.

### RNA Extraction

Total RNA was extracted from tumors and cell samples with the miRNeasy Mini Kit (Qiagen) according to the manufacturer's instructions.

### Real-Time Quantitative PCR

1 μg of extracted RNA from cells was reverse transcribed into cDNA using the Multiscribe reverse transcriptase kit provided by Applied Biosystems. Primers were designed using PrimerBank or adopted from published studies. Gene expression levels were measured using Platinum SYBR Green qPCR Supermix (Fisher Scientific) and Step One thermocycler (Applied Biosystem). Results from qPCR were normalized using the reference gene RPL32 and relative gene expression was quantified with the ΔΔCt method. Heatmaps describing gene expression fold changes were prepared using MeV software. cDNAs from tumors were prepared from 100 ng of RNA using Fluidigm Reverse Transcription Master Mix (Fluidigm PN 100-647297). Following a pre-amplification step (Fluidigm® PreAmp Master Mix and DELTAgene™ Assay kits) and exonuclease I treatment, samples diluted in Eva-Green® Supermix with Low ROX were loaded with primer reaction mixes in 96.96 Dynamic Array™ IFCs. Gene expression was then assessed on a Fluidigm BioMark HD instrument. Data were analyzed with real-time PCR analysis software (Fluidigm Corporation), and presented as relative gene expression according to the ΔΔCt method. Heat maps depicting fold changes of gene expression were prepared using MeV software.

### Fibrillar Collagen Imaging

Collagen in paraffin-embedded tumors was stained with picrosirius red using standard protocols as described (Girard et al., 2020). Tumor sections were analyzed by polarized light microscopy. Images were acquired under polarized illumination using a light transmission microscope (Zeiss PALM, at 10× magnification).

### In Vivo Experiments

Mouse experiments were carried out according to the Institutional Animal Care and the local ethical committee. 4×105 YUMNI1.7 cells were injected in both flanks of C57BL/6 mice. Tumors were measured with caliper and treatments were started when the tumors reached a volume of 0.1 cm3, after randomization of mice into control and test groups. Vemurafenib (30 mg/kg), Trametinib (0.3 mg/kg), and Nintedanib (50 mg/kg) were administered by oral gavage three times per week. Control mice were treated with vehicle only. Animals were sacrificed when the tumors reached a volume of 1 cm3. After animal sacrifice, tumors were dissected, weighed and snap-frozen in liquid nitrogen for RNA or protein extraction and immunofluorescence analysis (embedded in OCT from Tissue-Tek). Tumors for picrosirius red staining were fixed in formalin.

### Results

Nintedanib is a triple tyrosine kinase inhibitor first studied as an inhibitor of the angiogenesis-associated receptors PDGFR, VEGFR, and FGFR1 (Hilberg et al., 2008). Most importantly, PDGFR and FGFR1 have been involved in fibrotic processes. Consistently, Nintedanib has been approved for the treatment of idiopathic lung fibrosis (IPF) (Rivera-Ortega et al., 2018; Wollin et al., 2015). Its therapeutic efficacy in IPF is due to the inhibitory action on myofibroblast proliferation, differentiation, and collagen deposition. We recently reported that dedifferentiated mesenchymal MAPKi-resistant melanoma cells display markers observed in fibrotic diseases such as PDGFRβ expression and features proper of myofibroblasts including collagen-remodeling activities (Diazzi et al., 2020; Girard et al., 2020). Importantly, BRAF inhibition triggers in vivo this fibrotic-like phenotype with altered organization of the tumor ECM (Girard et al., 2020). With this in mind, we set out to analyze the effects of the anti-fibrotic drug Nintedanib in the context of early adaptation and resistance to MAPK inhibitors in melanoma cells.

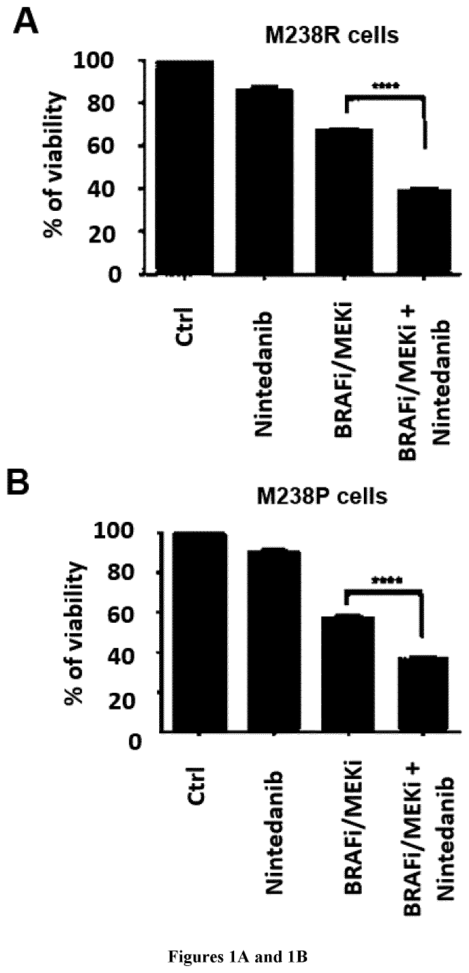

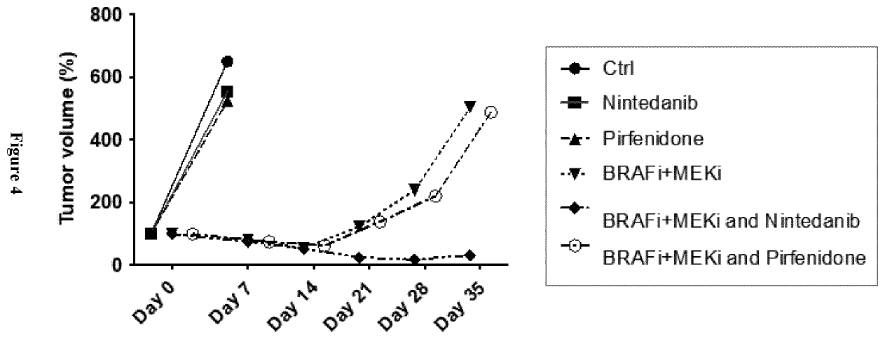

First, we sought to determine whether melanoma cells that acquired resistance could be re-sensitized to BRAFi/MEKi therapy upon co-treatment with Nintedanib. We chose M238R cells that had acquired resistance to BRAFi and expressed mesenchymal and myofibroblast markers (Girard et al., 2020). Resistant cells were obtained by chronic exposure of the melanoma BRAF-mutated sensitive M238P cells to the BRAFi Vemurafenib as described (Nazarian et al., 2010). Treatment with Nintedanib restored sensitivity of M238R cells to BRAF and MEK co-inhibition as shown by analysis of cell viability by crystal violet staining of attached cells (FIG. 1A). In BRAFi-sensitive M238P cells, we observed that BRAFi plus MEKi treatment decreased cell viability and Nintedanib potentiated significantly the inhibitory effect (FIG. 1B). Then, we evaluated the combination of Nintedanib with others different BRAFi/MEKi therapeutic combinations, in the presence or not of Nintedanib, on melanoma cell viability. BRAFi-resistant M238R cells were treated with vehicle, Nintedanib alone, a combination of BRAFi (Vemurafenib)/MEKi (Cobimetinib), a combination of BRAFi (Vemurafenib)/MEKi (Cobimetinib)/Nintedanib, a combination of BRAFi (Dabrafenib)/Trametinib or a combination of BRAFi (Dabrafenib)/Trametinib/Nintedanib for 3 days (FIG. 3).

Nintedanib alone had no effect on BRAFi-sensitive and resistant cell survival. Notably, Nintedanib at two different doses reverted the ECM and fibrotic-like signature of BRAFi resistant M238R cells (data not shown). In addition, Nintedanib strongly attenuated BRAFi-induced ECM-related signature and myofibroblast markers such as ACTA2, MYL9 and LOXL2 in M238P melanoma cells (data not shown). Nintedanib also reduced gene expression of phenotypic markers such as PDL1 and NGFR and prevented the downregulation of the differentiation marker MITF (data not shown). Note that PDL1 was also reduced in M238R cells treated with the anti-fibrotic drug (data not shown). These data indicate that Nintedanib can modulate the response of melanoma cells to oncogenic BRAF inhibition likely by preventing MAPKi-driven ECM remodeling and phenotype switching toward dedifferentiated mesenchymal and neural crest stem cell subpopulations that are known to be associated with non-mutational therapy tolerance.

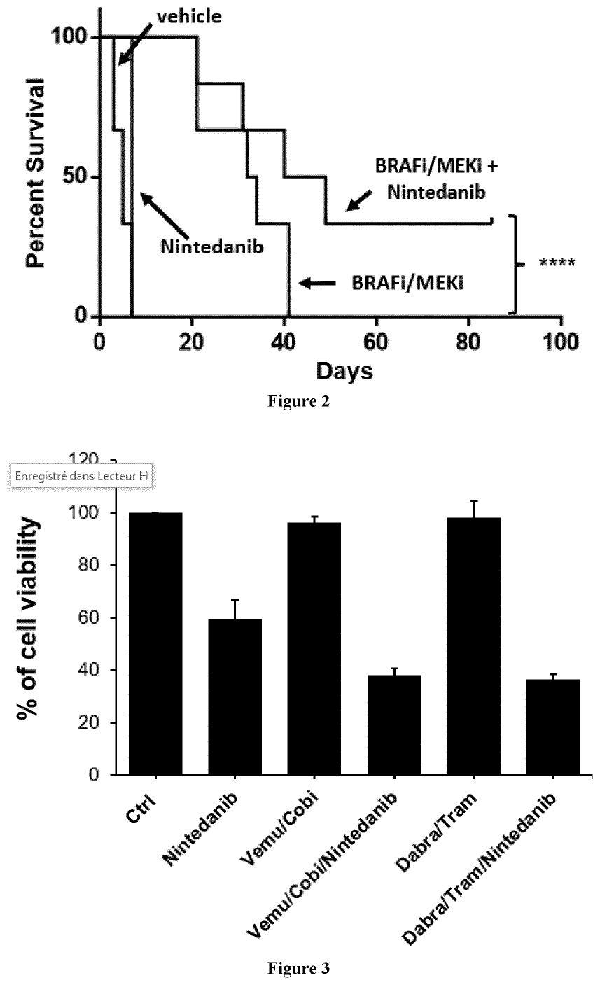

Next, we evaluated the combination of Nintedanib along with BRAFi plus MEKi in a syngenic model of murine YUMM1.7 Braf-mutant melanoma (Meeth et al., 2016). YUMM 1.7 cells were subcutaneously injected and tumors were treated with vehicle, Nintedanib, BRAFi (Vemurafenib)/MEKi (Trametinib), or BRAFi/MEKi and Nintedanib. Nintedanib did not display any anti-melanoma effect when administered alone, slightly slowing down tumor growth but not triggering tumor volume decrease. Administration of BRAFi plus MEKi initially reduced tumor growth but after three weeks of treatment, tumor growth resumed and 100% of tumors relapsed (data not shown). Importantly, combination of Nintedanib and MAPK-targeted therapies significantly delayed the relapse and led to complete remission in 33% of mice (2 out of 6) (FIG. 2). Overall, the combined treatment significantly improved mice survival without body weight loss or sign of toxicity throughout the study (data not shown). These results indicate that Nintedanib can delay the emergence of drug tolerant cells in vivo. As previously described in melanoma xenograft models, a profound ECM remodeling was observed in YUMM 1.7 tumors treated with MAPKi as revealed by fibers collagen picrosirius red staining (data not shown) and confirmed by real-time qPCR analysis of ECM, myofibroblast and pro-fibrotic markers (data not shown). These responses were suppressed by the co-administration of Nintedanib, indicating that Nintedanib counteracts the adverse effect of targeted agents on aberrant collagen deposition, a process potentially contributing to drug resistance and relapse. These results indicate that Nintedanib enhances the therapeutic effect of the MAPK-targeted therapy in a syngenic model of BRAF-mutant melanoma, likely through normalizing the stroma enriched in collagen fibers.

Then, we evaluated the impact of Nintedanib and Pirfenidone (an anti-fibrotic) in MAPK-targeted therapy efficacy and tumor relapse. We show that Nintedanib, but not Pirfenidone, enhances MAPK-targeted therapy efficacy and prevents tumor relapse of BRAFi/MEKi-treated YUMM1.7 melanoma (n=3) (FIG. 4).

In conclusion, our findings provide pre-clinical evidence that normalizing the pro-fibrotic stromal reaction driven by MAPK-targeted therapy can be exploited therapeutically to prevent and/or delay the emergence of therapy-resistant cells and tumor relapse. Our findings also provide a rationale for designing clinical trials with the clinically approved anti-fibrotic drug Nintedanib to enhance targeted therapies efficacy in BRAF-mutated melanoma patients.

### REFERENCES

Arozarena, I., and Wellbrock, C. (2019). Phenotype plasticity as enabler of melanoma progression and therapy resistance. Nat Rev Cancer 19, 377-391.

Diazzi, S., Tartare-Deckert, S., and Deckert, M. (2020). Bad Neighborhood: Fibrotic Stroma as a New Player in Melanoma Resistance to Targeted Therapies. Cancers (Basel) 12, 1364.

Girard, C.A., Lecacheur, M., Ben Jouira, R., Berestjuk, I., Diazzi, S., Prod'homme, V., Mallavialle, A., Larbret, F., Gesson, M., Schaub, S., et al. (2020). A Feed-Forward Mechanosignaling Loop Confers Resistance to Therapies Targeting the MAPK Pathway in BRAF-Mutant Melanoma. Cancer Res 80, 1927-1941.

Hilberg, F., Roth, G.J., Krssak, M., Kautschitsch, S., Sommergruber, W., Tontsch-Grunt, U., Garin-Chesa, P., Bader, G., Zoephel, A., Quant, J., et al. (2008). BIBF 1120: triple angiokinase inhibitor with sustained receptor blockade and good antitumor efficacy. Cancer Res 68, 4774-4782.

Marine, J.C., Dawson, S.J., and Dawson, M.A. (2020). Non-genetic mechanisms of therapeutic resistance in cancer. Nat Rev Cancer.

Meeth, K., Wang, J.X., Micevic, G., Damsky, W., and Bosenberg, M.W. (2016). The YUMM lines: a series of congenic mouse melanoma cell lines with defined genetic alterations. Pigment Cell Melanoma Res 29, 590-597.

Nazarian, R., Shi, H., Wang, Q., Kong, X., Koya, R.C., Lee, H., Chen, Z., Lee, M.K., Attar, N., Sazegar, H., et al. (2010). Melanomas acquire resistance to B-RAF(V600E) inhibition by RTK or N-RAS upregulation. Nature 468, 973-977.

Rambow, F., Marine, J.C., and Goding, C.R. (2019). Melanoma plasticity and phenotypic diversity: therapeutic barriers and opportunities. Genes Dev 33, 1295-1318.

Rivera-Ortega, P., Hayton, C., Blaikley, J., Leonard, C., and Chaudhuri, N. (2018). Nintedanib in the management of idiopathic pulmonary fibrosis: clinical trial evidence and real-world experience. Ther Adv Respir Dis 12, 1753466618800618.

Robert, C., Grob, J.J., Stroyakovskiy, D., Karaszewska, B., Hauschild, A., Levchenko, E., Chiarion Sileni, V., Schachter, J., Garbe, C., Bondarenko, I., et al. (2019). Five-Year Outcomes with Dabrafenib plus Trametinib in Metastatic Melanoma. N Engl J Med 381, 626-636.

Wollin, L., Wex, E., Pautsch, A., Schnapp, G., Hostettler, K.E., Stowasser, S., and Kolb, M. (2015). Mode of action of nintedanib in the treatment of idiopathic pulmonary fibrosis. Eur Respir J 45, 1434-1445.

1. A method for treating melanoma in a subject in need thereof comprising administering to said subject a therapeutically effective amount of: i) an inhibitor of BRAF, ii) an inhibitor of MEK and iii) an anti-fibrotic agent.

2. The method according to claim 1, wherein the melanoma is resistant melanoma.

3. The method according to claim 2, wherein, the melanoma is resistant to a treatment with inhibitors of BRAF mutations.

4. The method according to claim 2, wherein, the melanoma is resistant to treatment with inhibitors of MEK mutations.

5. The method according to claim 2, wherein, the melanoma is resistant to double-negative BRAF and NRAS mutations.

6. The method according to claim 2, wherein, the melanoma is resistant to treatment with an immune checkpoint inhibitor.

7. The method according to claim 1, wherein, i) the inhibitor of BRAF is Vemurafenib; ii) the inhibitor of MEK is Trametinib and iii) the anti-fibrotic agent is Nintedanib.

8. The method according to claim 1, wherein, i) the inhibitor of BRAF is Vemurafenib; ii) the inhibitor of MEK is Cobimetinib and iii) the anti-fibrotic agent is Nintedanib.

9. The method according to claim 1, wherein, i) the inhibitor of BRAF is Dabrafenib; ii) the inhibitor of MEK is Trametinib and iii) the anti-fibrotic agent is Nintedanib.

10. A pharmaceutical composition comprising i) an inhibitor of BRAF, ii) an inhibitor of MEK and iii) an anti-fibrotic agent.

11. (canceled)

12. (canceled)
